(19) United States
(12) Patent Application Publication
(10) Pub. No.: US 2020/0289480 A1
(43) Pub. Date: Sep. 17, 2020

(54) PERGOLIDE FOR USE IN THE TREATMENT OF EYE DISEASES ASSOCIATED WITH AN ELEVATED LEVEL OF VASCULAR ENDOTHELIAL GROWTH FACTOR (VEGF) AND PHARMACEUTICAL COMPOSITION CONTAINING PERGOLIDE

(71) Applicant: Maciej OSEKA, Duchnów (PL)

(72) Inventors: Maciej OSEKA, Duchnów (PL); Anna ZUBILEWICZ, Lublin (PL)

(21) Appl. No.: 16/651,179

(22) PCT Filed: Sep. 25, 2018
(86) PCT No.: PCT/IB2018/057378

§ 371 Date: Mar. 26, 2020

(30) Foreign Application Priority Data
Sep. 26, 2017 (PL) PL422978

(51) Int. Cl. A61K 31/4355; A61P 27/02

(52) U.S. Cl. CPC A61K 31/4355; A61P 27/02

(57) ABSTRACT

The object of the invention is pergolide for use in the treatment of eye diseases associated with an elevated level of vascular endothelial growth factor (VEGF) and a pharmaceutical composition for use in the treatment of eye diseases associated with an elevated level of vascular endothelial growth factor (VEGF), characterised in that it contains pergolide and a pharmaceutically acceptable carrier.

### FIELD OF THE INVENTION

The present invention relates to pergolide for use in the treatment of eye diseases associated with an elevated level of vascular endothelial growth factor (VEGF) and to a pharmaceutical composition for use in the treatment of eye diseases associated with an elevated level of vascular endothelial growth factor (VEGF) containing pergolide and a pharmaceutically acceptable carrier.

### PRIOR ART

Angiogenesis is a process of new blood vessels formation. Angiogenesis is accompanied by many physiological and pathological processes, such as the formation of collateral circulation in the heart muscle or the formation of vascularisation in solid tumours. The angiogenesis process is induced by vascular endothelial growth factor (VEGF). VEGF is released by many cells and tissues, including vascular endothelial cells, under the influence of various factors, e.g. inflammatory, and in response to hypoxia. Released VEGF acting through receptors results in vascular endothelium permeability and endothelial cell migration outside the lumen of the vessel. Migrating vascular endothelial cells arrange into tubular structures which are rudiments of new blood vessels. The VEGF-induced permeability of the natural barrier of endothelial cells in blood vessels is accompanied by permeation of fluid into the extravascular space, leading to oedema of the surrounding tissues.

In ophthalmology, pathological angiogenesis plays an important role in diseases such as AMD (age-related macular degeneration), in particular wet AMD, retinal artery occlusion, retinal vein thrombosis, diabetic macular oedema or diabetic retinopathy.

Diabetic macular oedema (DMO) occurs in patients with long-time diabetes and with more advanced form of diabetic retinopathy. It affects about 14% of diabetic patients. The occurrence of DME depends on the stage of retinopathy, as well as on the duration and type of diabetes. After 25 years of diabetes, DME affects about 30% of patients with type 1 and type 2 diabetes treated with insulin and about 15% of patients with type 2 diabetes treated with oral anti-diabetic drugs. Diabetic macular oedema is defined as the presence of fluid or hard exudates within 1 DD (optic nerve disc diameter) from the centre of the macula.

Diabetic retinopathy (DR) is currently the most frequent cause of blindness in developed countries. This is due to the increase in the incidence of diabetes in these countries. According to WHO, in 2002 diabetic retinopathy was the cause of blindness in 1.8 million people (4.8%) worldwide. In USA, it leads to blindness in 12-24 thousand patients with diabetes annually. From a clinical point of view, DR can take several forms, such as non-proliferative retinopathy, pre-proliferative retinopathy, diabetic maculopathy, proliferative retinopathy and advanced diabetic retinopathy. In the majority of these forms, retinal oedema occurs, and in case of proliferative forms the abnormal blood vessel growth is observed.

The occlusion of the central retinal artery or branches thereof is relatively rare. It is characterised by sudden, painless vision loss. Fast treatment leads to full or partial vision restoration. One of the complications in the central retinal artery occlusion is the formation of pathological blood vessels within eye tissues.

Retinal vein thrombosis relates to the occlusion of the lumen of the central retinal vein (central retinal vein occlusion—CRVO) or a branch thereof (branch retinal vein occlusion—BRVO). It leads to the vision deterioration at various levels. CRVO and BRVO are second most frequent retinal diseases of vascular origin. Ischaemic form is less frequent than the one without ischaemia. Retinal vein thromboses have the incidence of about 2 cases per 1000 people among individuals above 40 years of age, and above 5 cases per 1000 people among individuals above 64 years of age. Similarly as in the case of central retinal artery occlusion, the retinal vein thrombosis can result in late complications, such as neovascularisation of retina and other eye tissues, e.g. of angle or of iris.

Age-related macular degeneration (AMD) is the main cause of vision loss in people above 50 years of age. It affects 8.8% of population, more frequently women, and its prevalence increases with age, affecting 28% of people aged above 75. Worldwide, the number of affected individuals reaches 50 million. Due to the ageing of society, the problem of AMD keeps growing and about 10% of individuals above 45 years of age are at risk. 90% of AMD cases are so called dry AMD in which atrophic changes in the macula and gradual vision loss occur. 10% of patients with AMD have a wet AMD form in which neovascularisation of retina and choroid takes place (choroidal neovascularisation; CNV) and which in short time leads to significant vision impairment or vision loss.

Wet AMD is responsible for the majority of cases of vision loss. It is associated with retinal oedema which results from the blood-retinal barrier disruption and neovascularisation. The newly formed vessels are fragile and permeable, which aggravates the pathological retinal oedema and may lead to detachment of the retina. In case of changes which occur within the central area of the retina, called the macula, an affected individual experiences a central scotorna in theft field of vision, making every-day functioning impossible. Also, in the course of diabetes there is an increased VEGF release induced by the elevated blood sugar (hyperglycaemia) as well as by the action of so called advanced glycation end-products (AGE).

Currently, in order to reduce the oedema of eye tissues and to inhibit angiogenesis, inhibitors for VEGF or receptors thereof are used, such as ranibizumab (fragment of anti-VEGF-A recombinant humanised monoclonal antibody), bevacizumab (anti-VEGF-A recombinant humanized monoclonal antibody) and aflibercept (Fc fragment of VEGF receptor). These are administered in the form of repetitive intravitreal injections, typically at one- or two-month intervals. The administration of VEGF inhibitors in the form of injections requires sterile conditions for administration, limits the number of potential patients and entails the risk of complications.

Application No. P.415170 discloses the use of dopaminergic receptor type 2 agonists, especially cabergoline, in the treatment of eye diseases induced by the elevated level of vascular endothelial growth factor. Experimental studies showed that dopamine, acting by dopamine receptors D2, inhibits the permeability of blood vessels as well as proliferation and migration of endothelial cells. Without being bound by any theory, this result is probably achieved by the depletion of VEGF-2 receptors and the decrease in affinity of VEGF-2 receptors to VEGF. The above observations are supported by clinical data.

The inventors of the present invention unexpectedly found that pergolide has an inhibitory effect on VEGF release and action in angiogenesis model, which is more potent than for known agents, including other dopaminergic receptor type 2 agonists.

Pergolide, of the structural formulae presented below, belongs to the group of ergotamine derivatives of dopamine agonists used mainly in the treatment of Parkinson's disease. Pergolide stimulates dopaminergic receptors type D₁ and D₂, as well as serotoninergic type 5-HT_{1A}, 5-HT_{1B}, 5-HT_{2A}, 5-HT_{2B}, 5-HT_{2C}.

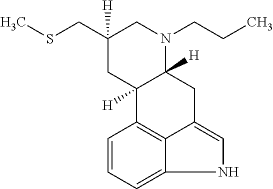 [SMILES: CCCN1C[C@H](CSC)C[C@@H]2c3cccc4[nH]cc(c34)C[C@H]21]

Thus, the object of the present invention is pergolide for use in the treatment of eye diseases associated with the elevated level of vascular endothelial growth factor (VEGF).

Preferably, the eye disease is age-related macular degeneration (AMD).

Preferably, age-related macular degeneration (AMD) is wet AMD.

Preferably, the eye disease is diabetic macular oedema and/or diabetic retinopathy.

Preferably, the eye disease is retinal vessels thrombosis and/or occlusion.

The object of the invention is also a pharmaceutical composition for use in the treatment of eye diseases associated with the elevated level of vascular endothelial growth factor (VEGF), characterised in that it contains pergolide and a pharmaceutically acceptable carrier.

Preferably, the eye disease is age-related macular degeneration (AMD).

Preferably, age-related macular degeneration (AMD) is wet AMD.

Preferably, the eye disease is diabetic macular oedema and/or diabetic retinopathy.

Preferably, the eye disease is retinal vessels thrombosis and/or occlusion.

### DESCRIPTION OF FIGURES

FIG. 1 Normal blood vessels in the eye (96 hours).

FIG. 2 Pathological blood vessels in the eye induced by CoCl₂. A higher number of vascular branches can be seen (96 hours).

FIG. 3 Inhibition of CoCl₂-induced abnormal vascular growth by bevacizumab (2.5 μg/ml; 96 hours).

FIG. 4 Normal ventral vessels (72 hours).

FIG. 5 CoCl₂-induced formation of abnormal ventral vessels (72 hours). A higher number of vascular branches can be seen.

FIG. 6 Inhibition of eye vessels formation by bromocriptine (2.5 μmol; 72 hours).

FIG. 7 Partial inhibition of abnormal blood vessels growth by bromocriptine (2.5 μmol; 72 hours).

FIG. 8 Full inhibition of abnormal ventral vessels growth by cabergoline (2.5 μmol; 72 hours).

FIG. 9 Partial inhibition of normal ventral vessels growth by pergolide (1 μmol; 72 hours).

### EXAMPLES

### Example 1

In studies on the effect of various substances on VEGF release and action, among others, a laboratory model using zebrafish (Danio rerio) is employed.

The study tested 3 substances with potential inhibitory activity towards VEGF release and action which belong to the group of dopaminergic receptor D₂ agonists—bromocriptine, cabergoline and pergolide.

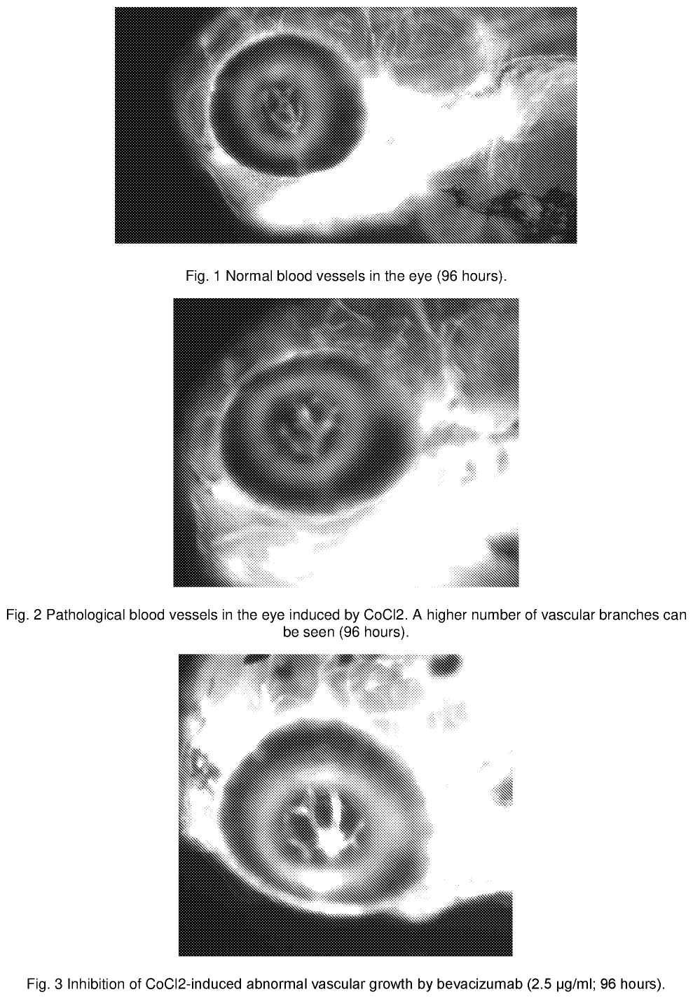

The study was conducted on the model using transgenic zebrafish embryos (Zebrafish Fli strain) with labelled blood system, illuminating under the fluorescent microscope. Photographic documentation was collected using Stereo Discover v8 fluorescent microscope, fluorescence 200c hxp, from Carl Zeiss Microscopy GmbH. During the study, the development of normal blood vessels and pathological blood vessels (abnormal angiogenesis) was observed. Abnormal angiogenesis was induced with the use of cobalt chloride (CoCl₂) in a dose of 5 mM which was administered to the culture medium. The study tested the inhibitory effect of bromocriptine, cabergoline and pergolide on the development of normal blood vessels, as well as the inhibition of abnormal angiogenesis in 5 increasing concentrations of these substances: 50 nmol, 100 nmol, 500 nmol, 1 μmol, 2.5 μmol, dissolved in dimethyl sulfoxide (DMSO) and administered to the culture medium. Any changes in the development of natural (FIG. 1) and pathological blood vessels (FIG. 2) were recorded 72 an 96 hours after the administration of a tested substance and a tested substance with CoCl₂, respectively. The level of inhibition of normal and pathological blood vessels development in tested embryos was compared to the activity of bevacizumab (Avastin) at the concentration of 2.5 μg/ml, which is an anti-VEGF recombinant humanised monoclonal antibody (FIGS. 3).

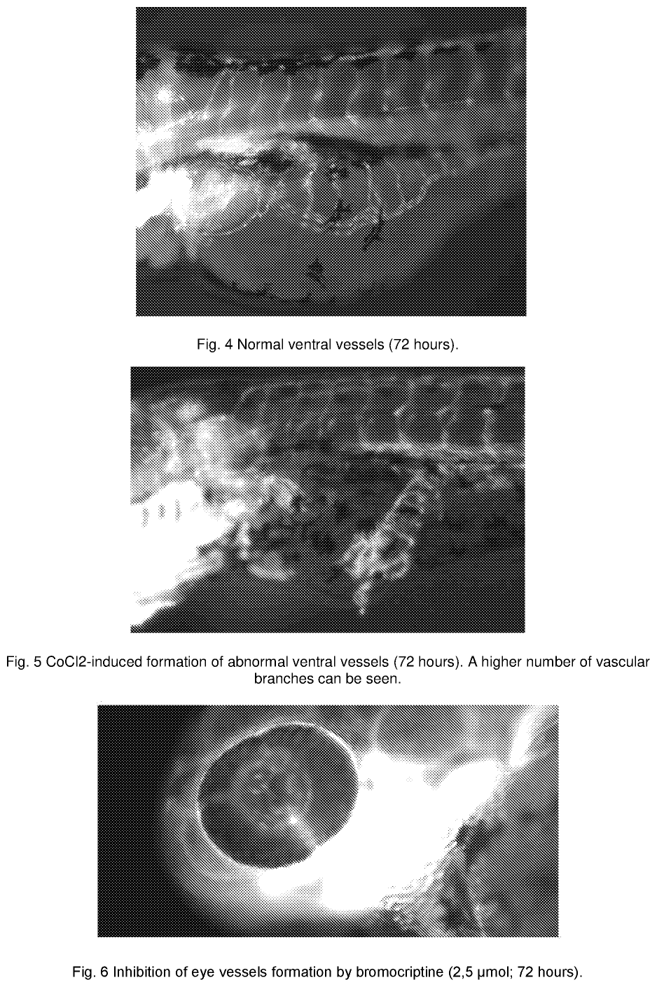

72 and 96 after CoCl₂ administration, the increase in the number of pathological eye blood vessels and the abnormal morphology thereof in the eyes (FIG. 2) as well as of ventral vessels (FIGS. 4 and 5) was observed.

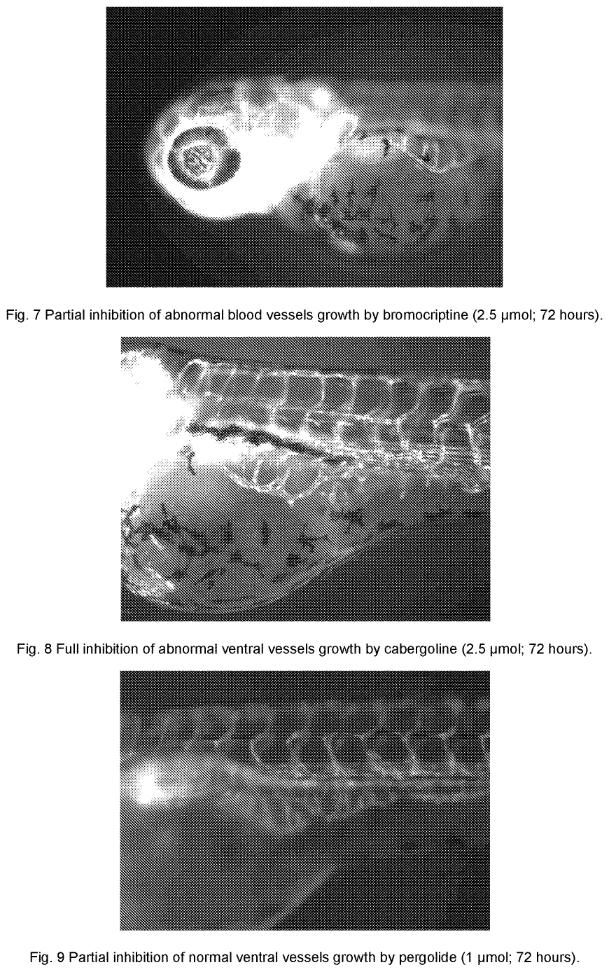

The use of bromocriptine at the maximum concentration of 2.5 μmol led to partial inhibition of growth of normal (FIG. 6) and pathological eye blood vessels (FIG. 7). For lower concentrations, no inhibitory effect on the development of normal and pathological blood vessels was observed.

The use of cabergoline at the maximum concentration of 2.5 μmol led to partial inhibition of growth of normal and full inhibition of growth of pathological eye blood vessels as well as ventral vessels (FIG. 8). The inhibitory effect of cabergoline was more potent compared to bromocriptine. Similarly as in the case of bromocriptine, no significant effect of cabergoline in lower concentrations on the development of normal and pathological blood vessels as well as ventral vessels was observed.

The use of pergolide at the concentration of 1.0 μmol led to partial inhibition of growth of normal and full inhibition of growth of pathological eye blood vessels as well as ventral vessels (FIG. 9). The use of pergolide at the concentration of 2.5 μmol led to full inhibition of growth of normal and abnormal eye blood vessels as well as ventral vessels.

Based on the above data, it was concluded that pergolide demonstrates potent inhibitory effect on the development of normal and pathological blood vessels, possibly suggesting its inhibitory effect on VEGF release and action.

1. Pergolide for use in the treatment of eye diseases associated with the elevated level of vascular endothelial growth factor (VEGF).

2. Pergolide for use according to claim 1, wherein the eye disease is age-related macular degeneration (AMD).

3. Pergolide for use according to claim 2, wherein age-related macular degeneration (AMD) is wet AMD.

4. Pergolide for use according to claim 1, wherein the eye disease is diabetic macular oedema and/or diabetic retinopathy.

5. Pergolide for use according to claim 1, wherein the eye disease is retinal vessels thrombosis and/or occlusion.

6. A pharmaceutical composition for use in the treatment of eye diseases associated with the elevated level of vascular endothelial growth factor (VEGF), characterised in that it contains pergolide and a pharmaceutically acceptable carrier.

7. The pharmaceutical composition for use according to claim 6, wherein the eye disease is age-related macular degeneration (AMD).

8. The pharmaceutical composition for use according to claim 7, wherein age-related macular degeneration (AMD) is wet AMD.

9. The pharmaceutical composition for use according to claim 6, wherein the eye disease is diabetic macular oedema and/or diabetic retinopathy.

10. The pharmaceutical composition for use according to claim 6, wherein the eye disease is retinal vessels thrombosis and/or occlusion.

11. A method for treating an eye disease associated with an elevated level of vascular endothelial growth factor, the method comprising administering a pharmaceutical composition comprising a therapeutically-effective amount of Pergolide to a patient having an eye disease associated with an elevated level of vascular endothelial growth factor.

12. The method according to claim 11 wherein the eye disease is age-related macular degeneration (AMD).

13. The method according to claim 12 wherein age-related macular degeneration (AMD) is wet AMD.

14. The method according to claim 11 wherein the eye disease is diabetic macular oedema and/or diabetic retinopathy.

15. The method according to claim 11 wherein the eye disease is retinal vessels thrombosis and/or occlusion.
